(19) United States
(12) Patent Application Publication
(10) Pub. No.: US 2010/0168518 A1
(43) Pub. Date: Jul. 1, 2010

(54) FLEXIBLE ENDOSCOPE DEVICE WITH VISUAL CONTROL AND PROCESS FOR STABILIZATION OF SUCH A DEVICE

(75) Inventors: Michel De Mathelin, Strasbourg (FR); Jacques Gangloff, Mulhausen (FR); Philippe Zanne, Roppenheim (FR); Laurent Ott, Limersheim (FR)

Correspondence Address: YOUNG & THOMPSON, 209 Madison Street, Suite 500, Alexandria, VA, 22314, US

(73) Assignees: UNIVERSITE DE STRASBOURG, Strasbourg (FR); CENTRE NATIONAL DE LA RECHERCHE SCIENTIFIQUE, Paris (FR)

(21) Appl. No.: 12/528,254

(22) PCT Filed: Feb. 25, 2008
(86) PCT No.: PCT/FR08/50312

§ 371 Date: Feb. 4, 2010

Related U.S. Application Data
(60) Provisional application No. 60/902,862, filed on Feb. 23, 2007.

(51) Int. Cl. A61B 1/005

(52) U.S. Cl. 600/118

(57) ABSTRACT

A flexible endoscope device includes an elongated flexible body having an end segment bearing or defining the endoscope head, and provided with an optical system that can be curved or bent in at least two mutually perpendicular directions, the elongated body being functionally connected at the other end thereof to a control element capable of controlling at least the movements and/or the arrangement of the end segment. The device includes automatic flexion and positioning elements of the end segment by visual control, the elements essentially including a video processing element for receiving the images or video signals provided by the optical system of the endoscope head using a computer element capable of carrying out visual control operations on the basis of the processed video signals and of transmitting control signals, and actuation members that are part of or associated with the control elements.

This invention relates to the field of equipment and instruments for medical use, more particularly equipment for operating rooms, medical investigation rooms, and medical offices, and it has as its object a flexible endoscopic device with visual control and a process for active stabilization of such an endoscope.

The flexible endoscopes are well known and have been used for many years in a number of medical applications, both for diagnostic purposes and for operating or operating assistance purposes.

Each procedure generally requires a specific flexible endoscope that is accordingly suitable and that is distinguished by its length, its diameter, the maneuverability of its invasive end, its accessories, the tools or instruments that it can integrate, . . . .

However, in all of the current flexible endoscopes, it is possible to systematically demonstrate at least the following three constituent parts:
An external manual control means, generally in the form of a control handle that is equipped with at least one, preferably two, control elements in the form of rollers or the like;
A supple and flexible elongated body, generally with a circular section and in which several longitudinal channels are provided for the passage of maneuvering instruments or cables;
A functional invasive end, in particular in the form of a flexible end segment of the elongated body (located opposite to the control handle), which carries or forms the head of the endoscope, is equipped with an optical system (for example of the CCD video camera type or the optical fiber bundle type) and that can be curved or bent in at least two preferably mutually perpendicular directions.

The channels of the elongated body also extend through this end segment at which they emerge.

The actuation of the head of the endoscope, i.e., the angle of deflection of the end segment, is monitored by maneuvering cables running along the elongated body and is connected to the control elements.

To navigate in the body of the subject, and thus to monitor the trajectory of the endoscope or to guide one or more instruments toward a target zone or element, the practitioner uses the images that are provided by the optical system, loaded into the active part (CCD camera, optical fiber). The lighting of the scene is also ensured by the endoscope via optical fibers that emerge at the end that forms the head. The practitioner actively acts on the endoscope by manipulating the elongated body (translation and rotation) by means of the handle and positively monitors the bending of the end segment in at least two orthogonal directions.

Once on the operating site, the practitioner can pass instruments such as, for example, clips, scissors, a hook, a bistoury, a laser fiber or else a needle, according to the type of intervention to be implemented, and can interchange them later without modifying the position of the endoscope.

The advantage of the use of the flexible endoscopes that are referred to above has been demonstrated recently, in an additional manner, within the framework of a new mini-invasive surgical technique called NOTES (Natural Orifice Transluminal Endoscopic Surgery) [see in particular “Per-Oral Transgastric Abdominal Surgery,” KO, C. W., KALLOO, A. N., Chinese Journal of Digestive Diseases 2006, 7: 67-70], which is currently being developed on an animal model and is in the stage of the first tests on man.

It consists in accessing the abdominal cavity by passing through a natural orifice (mouth, then stomach, anus then intestine, . . . ) and then through an internal membrane (gastric, intestinal, vaginal wall, . . . ) to accomplish treatments such as the tying of Fallopian tubes, a cholecystectomy or else a gastrojejunostomy. A first operation without a cutaneous incision on a human patient is described in the following publication: “Surgery without Scars: Report of Transluminal Cholecysstectomy in a Human Being,” J. Marescaux, B. Dallemagne, S. Perretta et al., Archives of Surgery, 2007, 142(9): 823-826.

The first transluminal surgical tests have been possible by the use of conventional flexible endoscopes accompanied by endoscopic tools that were used up until then in diagnostics and in treatments of pathologies present in gastric tracts. These instruments are not suitable for these new techniques, however.

One of the main problems encountered during the implementation of such flexible endoscopes arises from the fact that the flexible part of the endoscope cannot be controlled in a direct and absolute manner because of the absence of a rigid connection. Its shape and its position are defined by anatomical constraints (esophagus, stomach), i.e., by the anatomical structures with which the endoscope is in contact and by the effects of gravity.

Once the endoscope has penetrated the body of the subject, the practitioner does not know its shape. The practitioner must, for example, rely on the surrounding anatomical structures to guide the endoscope, whereby these structures are often themselves not stationary.

Once the target zone is reached, the practitioner is to combine different actions to carry out the desired movements.

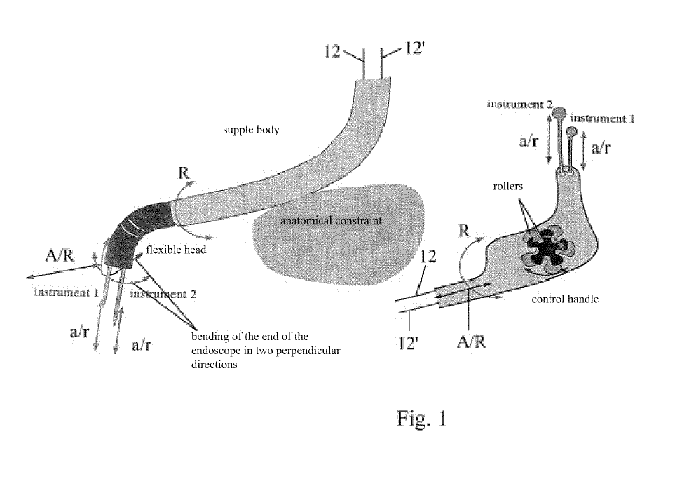

As FIG. 1 illustrates, the possible actions are the rotation of the rollers, the advance/retraction A/R of the endoscope in a direction that depends on the complete position of the body of the endoscope and the rotation R of the body of the endoscope. Since the position of the endoscope in the body of the patient is unknown, the movements of the body of the endoscope have effects that are difficult to predict on the level of the image. In addition, the rotation of the rollers produces an action that is certainly controllable but not very intuitive, which requires the practitioner to undergo long and difficult training.

The physiological movements of the organs and the patient (respiration, movements of the body) are sources of perturbations on the flexible endoscope. The compensation for these perturbations requires a very complex coordination between vision and movement of the endoscope. Good precision therefore requires a thorough training of the practitioner.

In addition, most of the current endoscopic instruments do not use articulations. The practitioner can only give them a movement of advance/retraction a/r that is independent of the movement of the endoscope. To give them mobility, unlike in the axis of the camera, the practitioner has to displace the head of the endoscope. Thus, it is not possible with the current endoscopic instruments to carry out independent movements with each of said instruments.

In some types of interventions, the number of mobilities to manage simultaneously to carry out an intervention requires—in practice—the action of several practitioners on the endoscopic system. These practitioners should then share a small work space around the control handle of the endoscope and demonstrate good coordination to implement medical or surgical movements.

The complexity of reduced manipulations and mobilities of tools do not make it possible for practitioners to carry out an operation as quickly and with the same dexterity as with more conventional operating techniques.

The object of this invention consists in proposing a solution that makes it possible to be free of physiological movements and more generally perturbations that are produced by the surroundings of the endoscope, and to facilitate the precise monitoring of the latter by the practitioner by relieving him of certain control and monitoring tasks, preferably without modifying the internal constitution of the endoscope or its mode of operation.

The invention should also make possible a specific use of a flexible endoscope by less experienced and/or less capable practitioners and in addition improve its facility of manipulation in a general way.

Motorized endoscopes have already been proposed by the documents FR-A-2 740 668, WO-A-9201414, and U.S. Pat. No. 4,941,454. However, these known solutions do not make it possible to respond to the object that this invention has set.

For this purpose, this invention has as its object a flexible endoscopic device that comprises an elongated supple body that has a flexible end segment that carries or forms the head of the endoscope, equipped with an optical system and able to be curved or bent in at least two mutually perpendicular directions, whereby said elongated body is connected functionally, at its other end, to a control means that can monitor at least the movements and/or the arrangement of said end segment, a device that is characterized in that it also comprises automatic means for orientation and positioning of the end segment by visual control, whereby these means essentially consist of a means of video processing receiving video images or signals supplied by the optical system of the head of the endoscope, by a computer means that can implement visual control operations on the basis of processed video signals and that supplies control signals and by actuating means that are part of or are associated with means of control that are able to monitor the end segment and receive control signals supplied by the computer means.

The invention also relates to a process for stabilization of a flexible endoscope by visual control, implementing a flexible endoscopic device that essentially comprises an elongated supple body that has a flexible end segment that carries or forms the head of the endoscope, equipped with an optical system and able to be curved or bent in at least two mutually perpendicular directions, whereby said elongated body is connected functionally, at its other end, to a control means that can monitor at least the movements and/or the arrangement of said end segment, a process that is characterized in that it consists in carrying out automatically, via automatic means of orientation and positioning of the end segment, processing of images supplied by the optical system of the head of the endoscope, operations of visual control on the basis of processed video signals, and the processing and delivery of control signals to actuating means that are part of or are associated with the control means.

Thus, a device and a process for automatic positioning of the head of the endoscope are proposed in the absence of positive intervention of the practitioner.

For this purpose, images taken by the optical system of the endoscope itself are used for the monitoring of the flexible end segment; this is done by means of a drive system for the control of this segment in combination with a visual control. A virtual connection between the head of the endoscope and the anatomical structure in question is thus produced, despite the physiological movements, the interaction of the instruments with the surroundings, and the manual movement of insertion/retraction of the endoscope.

Actually, the initial rough location of the end segment is obtained by manipulation of the endoscope in translation and in rotation by the practitioner, whereby the invention allows a monitoring of the fine positioning of said end segment, more specifically its orientation and its focusing on the target.

The basic principle that is implemented by the invention consists in, preferably in an iterative manner, estimating the pointing error of the endoscope relative to a target and developing a control, in real time, to lower and even eliminate this pointing error.

The invention will be better understood using the description below, which relates to a preferred embodiment, provided by way of nonlimiting example and explained with reference to the accompanying schematic drawings, in which:

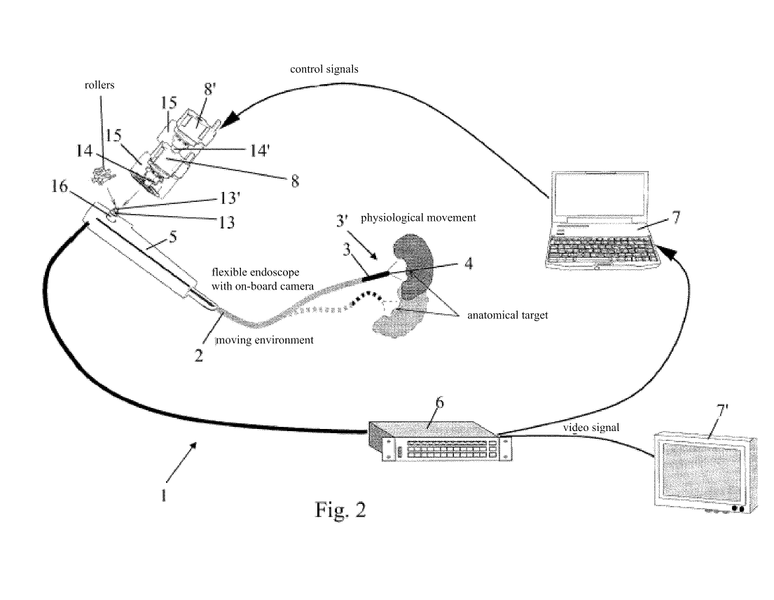

FIG. 2 is a schematic representation of a flexible endoscopic device according to the invention;

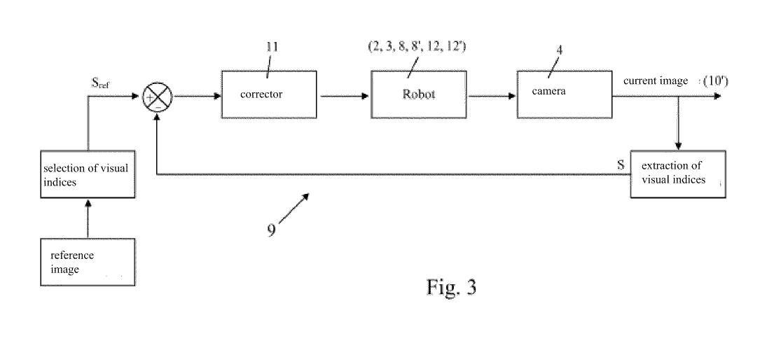

FIG. 3 is a functional schematic representation of a control loop by visual control with use of visual indices able to be implemented by the invention;

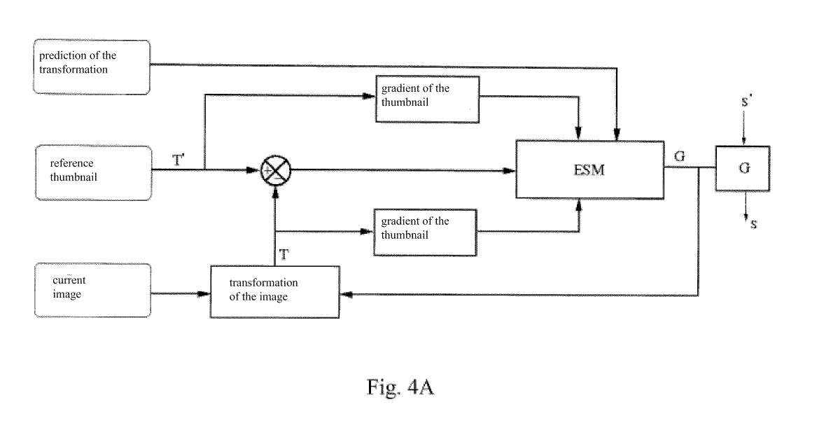

FIG. 4A is a functional schematic representation that illustrates the implementation of a visual tracking algorithm by second-order minimization that can be implemented within the framework of the invention;

FIG. 4B is a functional schematic representation of an active stabilization loop of the flexible endoscopic device that can be implemented by the invention, integrating the tracking algorithm shown in FIG. 4A;

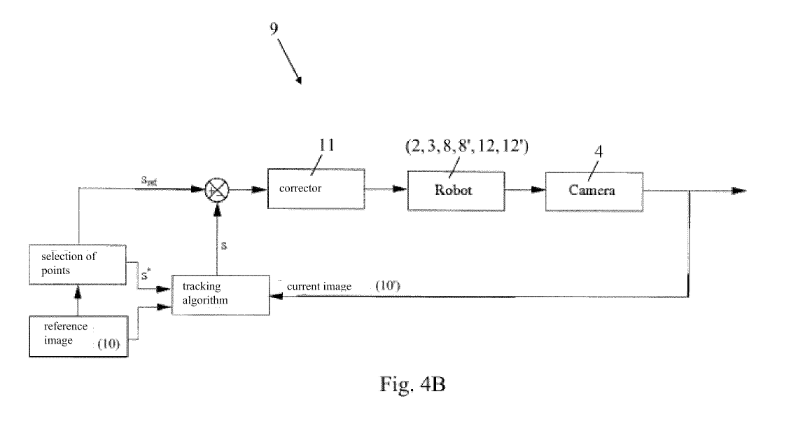

FIG. 5 is a block diagram of a modeling example of the control loop of FIGS. 3 and 4B;

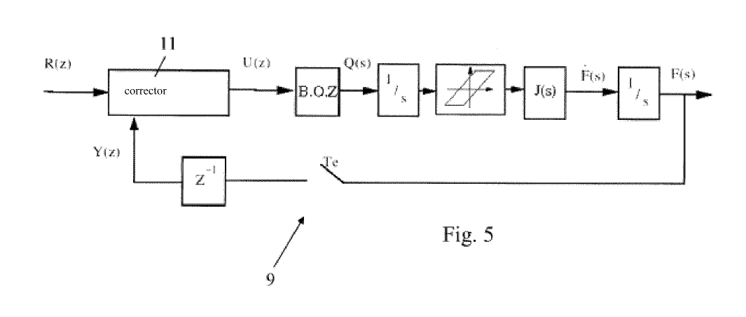

FIG. 6 is a perspective representation of the control means of the endoscopic device according to the invention, in the form of a motorized handle;

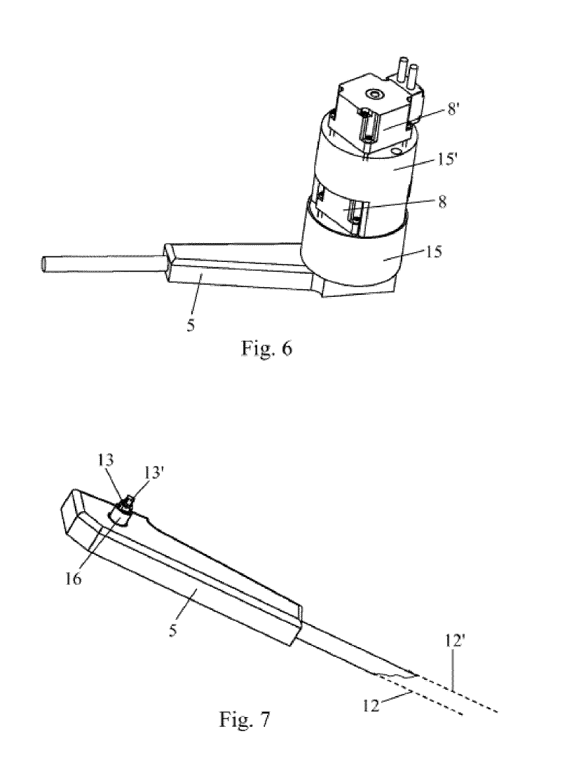

FIG. 7 is a perspective representation of a control handle of an endoscope, from which the manual and motorized control element is removed;

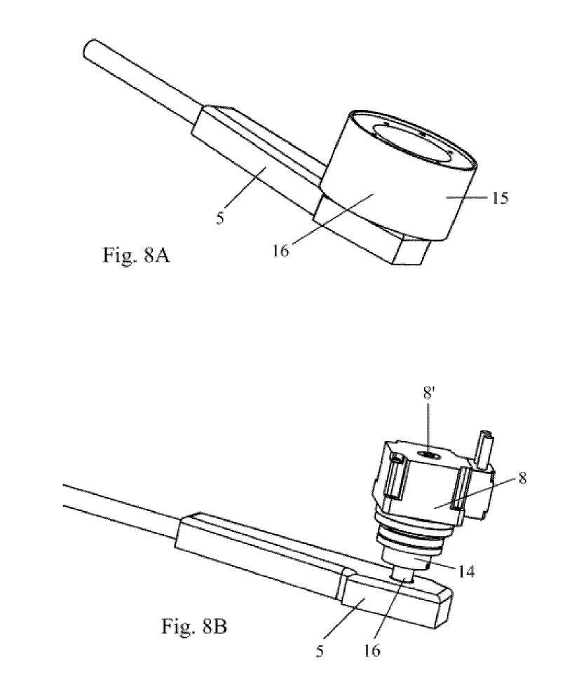

FIGS. 8A to 8C are perspective representations that successively illustrate the assembly of different components that form the motorized control means, namely respectively the support and attaching part, the first actuator, and the second actuator (only one component is shown per figure, whereby the representation of the other two components is omitted in each FIG. 8A to 8C to facilitate the perception of each component), and

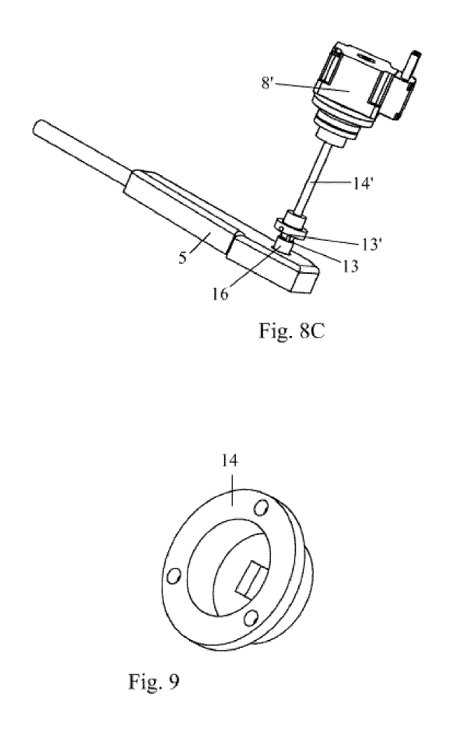

FIG. 9 is a detail perspective view of the transmission part that is associated with the motor forming the first actuator, shown in FIG. 8B.

FIG. 2 of the accompanying drawings shows a flexible endoscopic device 1 that comprises an elongated supple body 2 that has a flexible end segment 3 that carries or forms the head 3′ of the endoscope, equipped with an optical system 4 and able to be curved or bent in at least two mutually perpendicular directions, whereby the elongated body 2 is connected functionally, at its other end, to a control means 5 that can monitor at least the movements and/or the arrangement of said end segment 3.

According to the invention, the device 1 also comprises means of orientation and automatic positioning of the end segment 3 by visual control, whereby these means essentially consist of a video processing means 6 that receives video images or signals supplied by the optical system 4 of the head of the endoscope 3′, by a computer means 7 that can implement visual control operations on the basis of processed video signals and that supply control signals, and by actuating means 8, 8′ that are part of or are associated with the control means 5 that can monitor the end segment 3 (by bending) and that receive control signals provided by the computer means 7.

Of course, the video processing means 6 can constitute a separate unit (FIG. 2) or optionally be integrated with the computer means 7 in the same unit for processing and management.

As a variant, it can also be provided that a portion of the video processing means 6, or certain functions implemented by the latter, is/are located within the optical system 4.

The latter can also come in different variant embodiments, namely: an on-board video camera in the endoscope at the end segment 3 of the supple elongated body 2; a bundle of optical fibers that emerges at the end segment 3 and is connected to a video camera or a similar external device (fibroscope); an optical microfiber bundle that is mounted in the body 2 and is connected to an outside exploitation device (cellular fibroscopy device) or the like.

Thus, the head 3′ of the endoscope can either comprise the entire optical system 4 or the active part of said optical system 4 that transmits video signals or only one passive image acquisition element, connected to an offset functional unit, if necessary outside of the endoscope and optionally integrated in the computer means 7.

Likewise, at least one means 7′ for display of the video image supplied by the optical system 4 (for example, a CCD camera) can be provided.

According to a first significant characteristic of the invention, emerging in particular from FIGS. 3 and 4B of the accompanying drawings, the means 6, 7, 8, 8′, 12, 12′ for automatic bending and positioning of the end segment 3 are organized functionally for forming at least one control loop 9 that is designed to minimize the error or an analogous dissimilarity measure between, on the one hand, a visual element or a target piece of visual information extracted from a reference image 10, and, on the other hand, a visual element or a corresponding piece of visual information extracted from the current image 10′ supplied by the optical system 4 of the head of the endoscope 3′, whereby said error is exploited to deliver control signals to the actuating means 8, 8′ that monitor the movements and the positioning of said end segment 3 in at least one direction.

The computer means 7 then executes a digital command by visual control and contains a software model of the complex physical system formed by the endoscope, these mechanical control means and its drive system, whereby said modeling processes input signals formed by the reference image 10 and the current image 10′ and supplies control signals for the actuating means 8, 8′ at the output.

Thus, to obtain active stabilization, the images that are supplied by the optical system 4 and exploited in the form of video images are used to monitor the movements of the flexible endoscope, more particularly its head 3′ (see on this subject in particular: “A Tutorial on Visual Servo Control,” S. Hutchinson, G. D. Hager and P. I. Corke, IEEE Transactions on Robotics and Automation, Vol. 12, 5, pp. 651-670, 1996).

In a simplified manner, FIG. 3 shows a possible structure of a visual control loop 9 whose implementation is explained below in connection with the process according to the invention.

For its operation, such a visual control requires in advance the determination of at least one visual index in the image that is used in the adjustment.

In the case of an endoscopic device 1 with two degrees of freedom, i.e., an endoscope whose end segment 3 can be bent in two perpendicular directions, it is necessary to select two separate visual indices or a visual index with two components (for example, the coordinates of a point).

However, the working environment in which the head 3′ of the endoscope is located usually does not have particular visual indices such as points of interest, corners, contours of particular rigid shapes, straight lines or the like, and therefore does not directly make possible the application of the known procedures for extraction, comparison, tracking, and control, without installation of additional markers in the working environment.

To overcome this limitation, the invention advantageously proposes that the visual elements or a piece of visual information is/are selected from the group that is formed by visual patterns, image reductions, and the parts or zones of images that are obtained respectively from the reference image 10 and the current image 10′ provided by the optical system 4, if necessary after suitable processing of the latter.

In accordance with a preferred embodiment of the invention, it can be provided that the computer means 7 is able, by execution of a suitable program, to determine in real time, i.e., at least at the speed at which video images are supplied, an approximation, preferably by iterative processing, of the transformation to be applied to the current image 10′ that ends in a minimization of a cost function between a reduced image or a thumbnail T* obtained from the reference image 10 and a reduced image or thumbnail T that is obtained from the current image 10′ that is provided by the head 3′ of the endoscope and processed by the current approximated transformation.

Thus, the information that is useful for visual control is based on an algorithm of the “visual tracking of reference thumbnail” type. The reference zone is learned during the manual selection, by the practitioner, of the target zone to stabilize. The tracking of this zone is then ensured by minimizing a measure of dissimilarity between the reference thumbnail T* and the current thumbnail T.

In this document, “reference” image is defined as the image in which the practitioner identified the anatomical target. It normally consists of an initial or basic image provided by the video means that are connected to the endoscope 2 or optionally a stored image, for example a real image of the anatomical environment of the target acquired by selection.

According to a first variant embodiment, the minimization algorithm that is implemented by the computer means 7 produces a parametric minimization of the quadratic error between the visual elements or a piece of visual information T* and T obtained respectively from the reference image 10 and from the current image 10′, whereby the approximated transformation is a plane transformation, for example, of homographic type.

Thus, the minimization algorithm can be derived from, for example, an ESM-type algorithm, as described in particular in the publication “Improving Vision-Based Control Using Efficient Second-Order Minimization Techniques,” MALIS, E., IEEE Int. Conf. on Rob. and Aut., pages 1843-1848, 2004.

Such an algorithm makes possible, as indicated, a parametric minimization of the quadratic error between T and T*.

The configuration of the implementation of this minimization algorithm is shown in FIG. 4A and a more complete description emerges from: “Real-Time Image-Based Tracking of Planes Using Efficient Second-Order Minimization,” S. Benhimane and E. Malis, IEEE/RSJ, Int. Conf. on Intelligent Robot and System, pages 943-948, 2004.

In this context, the transformation estimated by the computer means 7 is a plane transformation, in particular homographic, making it possible to locate each pixel or point of the visual element individually and to use it as a visual index.

To implement a stabilization of the head of the endoscope 3′ with regard to a moving anatomical target, the visual control loop 9 as shown in FIG. 4B, which integrates the algorithm according to FIG. 4A, is proposed.

According to a second variant embodiment of the invention, it can also be provided that the minimization algorithm implemented by the computer means 7 produces a statistical minimization of a measure of dissimilarity between histograms that are obtained from visual elements or a piece of visual information T* and T obtained respectively from the reference image 10 and from the current image 10′, whereby the approximated transformation consists of a translation in the image plane and a zoom (reduction or enlargement).

Thus, in accordance with this second variant, the minimization algorithm can be derived from the one that is known under the algorithm designation of “mean-shift,” as described in, for example, “Kermel-Based Object Tracking,” D. Comanicin et al., Patterne Analysis and Machine Intelligence, IEEE Transactions on, Volume 25, May 5, 2003, pages: 564 to 577.

As FIG. 6 illustrates in connection with an above-mentioned tracking or minimization algorithm (above-mentioned ESM algorithm or “mean shift” algorithm), the vector of the pre-parameterized variable s_{ref} contains the image data in which the anatomical target is to be stabilized. This point is selected initially by the practitioner on the reference image 10 and can be modified in the current image 10′. The practitioner also specifies the point of interest of the anatomical target to be stabilized s* in the reference thumbnail T*. One of the above-mentioned follow-up algorithms is then used to reconstruct the transformation between the current thumbnail T and the reference thumbnail T*, whereby this transformation is then used to determine the vector s that contains the coordinates of the point to be stabilized in the current image 10′.

According to a characteristic of the invention, the automatic positioning means 6, 7, 8, 8′ are organized in a two-dimensional visual control loop and comprise two independent actuators 8 and 8′ that each monitor the movements and the positioning of the end segment 3 in one of the two mutually perpendicular directions that correspond to the two directions of the plane current image 10 supplied by the head of the endoscope 3′.

Optionally, an independent control loop 9 is provided for each actuator means 8, 8′ according to the nature of the system.

The monitor or corrector 11 that is part of the control loop 9 can be selected from the group that is formed by the monitors of the proportional type, of the proportional/integral/derivative type, and of the predictive or repetitive type.

Thus, when it is necessary to initiate a compensation or a cancellation of periodic movements, it is possible to use either a monitor 11 of the repetitive type as described in, for example: “Analysis and Synthesis of Discrete-Time Repetitive Controllers,” M. Tomizuka et al., American Society of Mechanical Engineers Journal of Dynamic Systems, Measurement and Control, 111, 353-358, 1989; or an R-GPC-type monitor as described in, for example: “Model Predictive Control for Pensation of Cyclic Organ Motions in Tele-Operated Laparoscopic Surgery,” J. Gangloff et al., Control Systems Technology, IEEE Transactions on, Volume 14, Issue 2, March 2006, pages: 235-246.

Whenever the perturbation can be predicted (for example in the case of artificial respiration), the monitor 11 can also integrate a model of the perturbation, such as, for example, in the predictive monitor described in the publication by E. F. Camacho, C. Bordons, “Model Predictive Control,” ISBN 3-540-76241-8.

In accordance with another characteristic of the invention, emerging from FIGS. 2, 6 and 8, the control means 5 comprises two concentric control shafts 13 and 13′ that are each associated with a control cable 12, 12′ that circulates along the elongated body 2 and is connected to the end segment 3 to monitor the bending or arching of said segment 3 in one of the two directions of mutually perpendicular bending, whereby the outside shaft 13 is in a drive relationship with a first actuating means 8 in the form of a motor with a hollow shaft 8″ by means of a transmission part 14 that is detached in a central region that extends into the continuation of the hollow shaft 8″ of said motor 8, and whereby the inside shaft 13′ is in a drive relationship, by means of an elongated drive part 14′, with a second actuating means 8′ in the form of a motor that is aligned with the first motor 8 along their axes of rotation, whereby said elongated drive part 14′ passes through the first motor 8 at its hollow shaft 8″.

Advantageously, the two motors 8 and 8′ are assembled mechanically with one another to form a drive system that is mounted on the handle 5 that forms the control means, by means of a support and attaching part 15, if necessary in the form of a unit with a removable mechanical connection, thus allowing an interchangeability with the manual control elements (FIG. 2).

The non-limiting drive system embodiment of the control of a flexible endoscope, illustrated in FIGS. 2 and 6 to 8, relates more particularly to a 13801 PKS-type endoscope of the KARL STORZ Company.

The drive system solution proposed by the invention makes it possible to preserve the existing internal structure of the endoscope by recommending that the manual control rollers be replaced by the actuators 8 and 8′.

The latter come, for example, in the form of harmonic control motors (for example of the FHA-8C type), controlled by speed-loop servocontrollers (for example of the Harmonic Drive SC-610 type). Said motors are connected directly to the control shafts 13 and 13′ by rigid mechanical connection and with neither clutch nor articulation, so as to prevent any play as much as possible.

A modeling example of the visual control loop of such a regulated and motorized system is shown in the form of a block diagram in FIG. 5 of the accompanying drawings.

In connection with this figure, it is possible to note that whereby the endoscopic device 1 is a mechanical system with two degrees of freedom, the two coordinates x and y of a visual index of the point type in the image is an adequate retroaction variable for monitoring said system.

The velocity of the visual index F(s) in the image is connected to the velocity of the actuators Q by a 2×2 interaction matrix that the inventors selected to estimate around the working configuration in a preliminary phase.

The velocity vector of the actuators Q(s)* is sent as a reference to the speed loops of the power amplifiers that control the motors of the actuators 8 and 8′. Since the bandwidth of said speed loops is considerably larger than the sampling frequency of the visual control loop 9, the dynamics of the actuators can be disregarded and thus Q(s) is approximately equal to Q(s)*.

As FIG. 5 shows, the primary branch of the loop comprises the following elements, in this order: corrector/zero-order blocker B.O.Z.)/1/s integrator/hysteresis module (the total of the degrees of plays of the device 1)/interaction matrix J(s)/1/s integrator and the retroaction branch comprises a delaying element Z⁻¹ that corresponds to the image acquisition time.

The discrete temporal transfer function of the system shown in block diagram form in FIG. 5 is then:

$$G ( z ) = \frac{Y ( z )}{U ( z )} = {z}^{- 1} ( 1 - z ) Z \{ \frac{J}{{s}^{2}} \} = J \frac{{T}_{e} \cdot {z}^{- 2}}{1 - {z}^{- 1}}$$

in which Z represents the operator of the transformation at z.

Within the framework of a practical mechanical embodiment of the invention, and in accordance with the practical embodiment illustrated in the above-mentioned FIGS. 2 and 6 to 8, the two control shafts have square sections and the inside shaft 13′ extends in a projecting manner through the outside shaft 13, whereby these two shafts 13 and 13′ are surrounded by a threaded sleeve 16 with a circular section, through which these two shafts 13 and 13′ extend and which is made integral in a rigid manner with the structure of the handle 5.

A cylindrical, hollow support part 15, advantageously made integral by screwing with the sleeve 16 (for example by means of a threaded opening that becomes engaged with said sleeve) and resting against the housing of the handle 5, implements the mounting of the motors 8, 8′ on the latter, according to a superposed configuration.

This part 15 is also configured so that it ensures an axial alignment of the motors 8 and 8′ with the concentric control shafts 13 and 13′.

This part 15 directly supports the lower motor 8 and indirectly the upper motor 8′, the latter by means of an additional support part 15′, made integral simultaneously with said part 15 and said motor 8′ (FIGS. 2 and 6).

As a variant on the above-mentioned mode by which the support part 15 engages with the handle 5, an attachment mode can also be provided by a removable housing, allowing an easy interchangeability between the manual control rollers and the drive system unit 8, 8′, 14, 14′, 15, 15′.

The part 14 for transmission of the movement between the lower motor 8 and the outside control shaft 13 has a detached central region (in the axial direction of the two motors in the mounted state) and is equipped at its bottom with a square cut-out for engagement that is adapted to the outside shaft 13 and is equipped at its upper part with sites for engagement with the output flange of the lower hollow-shaft motor 8 (for example, connection of the lugs/openings type).

As FIG. 8C shows in particular, the drive part 14′ that ensures the transmission of the movement between the upper motor 8′ and the inside shaft 13′ comprises a lower connecting part (with an opening with a square cross-section) that is designed to engage on said shaft 13′ and an upper connection interface engaged with the output flange of the upper motor 8′ (for example, by means of a lugs/openings connection).

The upper motor 8′ is kept in axial alignment with the lower motor 8 and the control shafts 13 and 13′ by means of the additional support part 15′.

According to a variant, not shown, the actuating means 8, 8′ can also come in the form of motors that are directly housed in the handle that forms the control means 5 and in a drive relationship each with a cable for control of the deformation by bending of the end segment 3.

Within the framework of this last variant, the control means 5 can also comprise at least one element that can be manipulated by the practitioner, such as a roller, a lever or the like, able to control at least one actuator 8, 8′, in an alternative or superposed way relative to the automatic command by visual control, whereby said element(s) is/are mounted at the handle 5, or offset relative to the latter.

Thus, it is possible to use an assisted manual command, which can be superposed on the automatic command by visual control, or to operate in a decoupled manner relative to the latter.

In the case of the implementation of on-board rollers on the handle 5, the practitioner will find the current manipulation configuration while benefiting from the advantages of the invention.

This invention also has as its object a process for stabilization of a flexible endoscope by visual control.

This process uses a flexible endoscopic device 1 that essentially comprises an elongated supple body 2 that has a flexible end segment 3 that carries or forms the head 3′ of the endoscope, equipped with an optical system 4 and able to be curved or bent in at least two mutually perpendicular directions, whereby said elongated body 2 is connected functionally, at its other end (opposite, or segment 3), to a control means 5 that can monitor at least the movements and/or the arrangement of said end segment, more particularly a device as described above.

This process is characterized in that it consists in automatically carrying out, via means 6, 7, 8 and 8′ for automatic bending and positioning of the end segment 3 that is organized functionally in at least one control loop 9, a processing of images supplied by the optical system 4 of the head 3′ of the endoscope, visual control operations on the basis of processed video signals, and the processing and delivery of control signals to actuating means 8, 8′ that are part of or are associated with the control means 5.

According to one characteristic of the invention, the control operations essentially consist in minimizing the error or an analogous dissimilarity measure between, on the one hand, a visual element or a target piece of visual information extracted from a reference image 10, and, on the other hand, a visual element or a corresponding piece of visual information extracted from the current image 10′ that is provided by the optical system 4 of the head of the endoscope 3′, whereby said error is exploited to deliver control signals to actuating means 8, 8′ monitoring the movements and the positioning of said end segment 3 in at least one direction.

Preferably, the visual elements or a piece of visual information is/are selected from the group that is formed by the visual patterns, image reductions, and parts or zones of images that are obtained respectively from the reference image 10 and the current image 10′ supplied by the optical system 4, if necessary after suitable processing of the latter.

According to one advantageous embodiment of the invention, the process consists in, within the framework of the control operations carried out by the computer means 7, determining in real time, i.e., at least at the speed at which video images are supplied, an approximation, preferably by an iterative processing, of the transformation to be applied to the current image 10′ that ends in a minimization of a cost function between a reduced image or a thumbnail T* obtained from the reference image 10 and a reduced image or thumbnail T obtained from the reference image 10′ supplied by the head 3′ of the endoscope and processed by the current approximated transformation.

According to a first variant of the process, the latter can consist in implementing a minimization algorithm that produces a parametric minimization of the quadratic error between the visual elements or a piece of visual information T* and T obtained respectively from the reference image 10 and from the current image 10′, whereby the approximated transformation is a plane transformation, for example, of homographic type.

According to a second variant of the process, the latter can consist in implementing a minimization algorithm that carries out a statistical minimization of a measure of dissimilarity between histograms that are obtained from visual elements or a piece of visual information T* and T obtained respectively from the reference image 10 and from the current image 10′, whereby the approximated transformation consists of a translation in the image plane and a zoom.

Advantageously, it is provided that the movements and the positioning of the end segment 3′ are monitored by two independent actuators 8 and 8′ each associated with one of the two perpendicular directions corresponding to two directions of the plane current image 10′ supplied repeatedly by the head of the endoscope 3′.

In connection with FIG. 3, the practical implementation of the process according to the invention can comprise two successive phases, namely a phase for manual parameterization of a setpoint s_{ref} and a phase for automatic video-rate adjustment of the error: e=s−s_{ref}, where s is the current value that corresponds to the setpoint, toward zero.

For the practitioner, the parameterization phase can consist in determining the setpoint s_{ref} of the control loop 9 in the form of suitable visual indices (for example, x, y coordinates of a point) that are selected from a reference image 10, for the purpose of monitoring the various degrees of freedom of the flexible endoscope, in particular its free end 3.

Then, the automatic adjustment phase can consist in repeating the following stages in a cyclic manner:

a) Extraction in the current image 10′ of current visual indices s corresponding to visual indices s_{ref} forming the setpoint and selected from the reference image 10;

b) Calculation of the error e=s−s_{ref};

c) Calculation of the control signals to be applied to actuators 8 and 8′, in particular speed signals, to reduce the error e;

d) Dispatch of control signals;

e) Acquisition of a new current image 10′ by means of the optical system and optional processing of this image;

f) Return to stage a).

So as to make the control more robust and to make target detection more efficient, it can also be provided to install in advance an artificial reference in the immediate vicinity of the anatomical target.

An example of practical implementation of the invention as well as the advantages that are obtained by the latter are described in the following publication: “Problématique de l'assistance robotique à la chirurgie transluminale endoscopique [Problems of Robotic Assistance with Endoscopic Transluminal Surgery],” Files of the Surgetica Conference 2007; Computer-Aided Medical Interventions; September 2007, Chambéry, France, whose content is integrated in this document by reference.

Of course, the invention is not limited to the embodiment that is described and shown in the accompanying drawings. Modifications are possible, in particular from the standpoint of the composition of the various elements or by substitution of equivalent techniques, without thereby exceeding the field of protection of the invention.

1-24. (canceled)

25. Flexible endoscopic device
whereby this device (1) comprises an elongated supple body (2) that has a flexible end segment (3) that carries or forms the head of the endoscope, equipped with an optical system (4) and able to be curved or bent in at least two mutually perpendicular directions, whereby said elongated body (2) is connected functionally, at its other end, to a control means (5) that can monitor at least the movements and/or the arrangement of said end segment,
whereby this device (1) also comprises automatic means for orientation and positioning of the end segment (3) by visual control, whereby these means essentially consist of a means of video processing (6) receiving video images or signals supplied by the optical system (4) of the head of the endoscope (3′), by a computer means (7) that can implement visual control operations on the basis of processed video signals and providing control signals, and by actuating means (8, 8′) that are part of or are associated with control means (5) that are able to monitor the end segment (3) and receive control signals supplied by the computer means (7),
whereby the means (6, 7, 8, 8′, 12, 12′) for automatic bending and positioning of the end segment (3) are organized functionally for forming at least one control loop (9) that is designed to minimize the error or an analogous dissimilarity measure between, on the one hand, a visual element or a target piece of visual information extracted from a reference image (10), and, on the other hand, a visual element or a corresponding piece of visual information extracted from the current image (10′) supplied by the optical system (4) of the head of the endoscope (3′), whereby said error is exploited to deliver control signals to the actuating means (8, 8′) monitoring the movements and the positioning of said end segment (3) in at least one direction,
device (1), characterized in that the computer means (7) is able, by execution of a suitable program, to determine in real time, i.e., at least at the speed at which video images are supplied, an approximation, preferably by iterative processing, of the transformation to be applied to the current image (10′) that ends in a minimization of a cost function between a reduced image or a thumbnail (T*) obtained from the reference image (10) and a reduced image or thumbnail (T) that is obtained from the current image (10′) that is provided by the head (3′) of the endoscope and processed by the current approximated transformation.

26. Device according to claim 25, wherein the visual elements or piece of visual information is/are selected from the group that is formed by the visual patterns, the reductions of images and the parts or zones of images that are obtained respectively from the reference image (10), in the form of an initial image or a basic image supplied by the optical system (4) or a real image of the anatomical environment of the target acquired by selection, and the current image (10′) supplied by the optical system (4), if necessary after suitable processing of the latter.

27. Device according to claim 25, wherein the minimization algorithm that is implemented by the computer means (7) produces a parametric minimization of the quadratic error between the visual elements or a piece of visual information (T* and T) obtained respectively from the reference image (10) and from the current image (10′), whereby the approximated transformation is a plane transformation, for example, of homographic type.

28. Device according to claim 25, wherein the minimization algorithm implemented by the computer means (7) produces a statistical minimization of a measure of dissimilarity between histograms that are obtained from visual elements or a piece of visual information (T* and T) obtained respectively from the reference image (10) and from the current image (10′), whereby the approximated transformation consists of a translation in the image plane and a zoom.

29. Device according to claim 25, wherein the automatic positioning means (6, 7, 8, 8′) are organized in a two-dimensional visual control loop and comprise two independent actuators (8 and 8′) that each monitor the movements and the positioning of the end segment (3) in one of the two mutually perpendicular directions that correspond to the two directions of the plane current image (10) supplied by the head of the endoscope (3′).

30. Device according to claim 25, wherein an independent control loop is provided for each actuating means (8, 8′).

31. Device according to claim 25, wherein the monitor or corrector (11) that is part of the control loop (9) is selected from the group that is formed by the monitors of the proportional type, of the proportional/integral/derivative type, and of the predictive or repetitive type.

32. Device according to claim 25, wherein the control means (5) comprises two concentric control shafts (13 and 13′) that are each associated with a control cable (12, 12′) that circulates along the elongated body (2) and is connected to the end segment (3) to monitor the bending or the arching of said segment (3) in one of the two mutually perpendicular bending directions, whereby the outside shaft (13) is in a drive relationship with a first actuating means (8) in the form of a motor with a hollow shaft (8″) by means of a transmission part (14) that is detached in a central region that extends into the continuation of the hollow shaft (8″) of said motor (8), and whereby the inside shaft (13′) is in a drive relationship, by means of an elongated drive part (14′), with a second actuating means (8′) in the form of a motor that is aligned with the first motor (8) along their axes of rotation, whereby said elongated drive part (14′) passes through the first motor (8) at its hollow shaft (8″).

33. Device according to claim 32, wherein the two motors (8 and 8′) are assembled mechanically with one another to form a drive system unit that is mounted on the handle (5) that forms the control means, by means of a support and attaching part (15), if necessary in the form of a unit with a removable mechanical connection.

34. Device according to claim 25, wherein the actuating means (8, 8′) come in the form of motors that are housed directly in the handle that forms the control means (5) and each in drive relationship with a cable for controlling deformation by bending of the end segment (3).

35. Device according to claim 33, wherein it comprises at least one element that can be manipulated by the practitioner, such as a roller, a lever or the like, able to control at least one actuator (8, 8′) in an alternative or superposed way relative to the automatic command by visual control, whereby said element is either mounted at the handle (5) or offset relative to the latter.

36. Process for stabilization of a flexible endoscope by visual control,
whereby said process implements a flexible endoscopic device (1) that comprises
essentially an elongated flexible body (2) that has a flexible end segment (3) that carries or forms the head of the endoscope, equipped with an optical system (4) and able to be curved or bent in at least two mutually perpendicular directions, whereby said elongated body is connected functionally, at its other end, to a control means (5) that can monitor at least the movements and/or the arrangement of said end segment,
whereby said process consists in producing automatically, via automatic means (6, 7, 8 and 8′) for orientation and positioning of the end segment (3) organized functionally in at least one control loop (9), a processing of images supplied by the optical system (4) of the head (3′) of the endoscope, visual control operations on the basis of processed video signals and the processing and delivery of control signals to actuating means (8, 8′) that are part of or are associated with the control means (5),
whereby the control operations essentially consist in minimizing the error or an analogous dissimilarity measure between, on the one hand, a visual element or a target piece of visual information extracted from a reference image (10), and, on the other hand, a visual element or a corresponding piece of visual information extracted from the current image (10′) supplied by the optical system (4) of the head of the endoscope (3′), whereby said error is exploited to deliver control signals to the actuating means (8, 8′) that monitor the movements and the positioning of said end segment (3) in at least one direction,
process wherein it consists in, within the framework of the control operations implemented by the computer means (7), determining in real time, i.e., at least at the speed at which video images are supplied, an approximation, preferably by an iterative processing, of the transformation to be applied to the current image (10′) that ends in a minimization of a cost function between a reduced image or a thumbnail (T*) obtained from the reference image (10) and a reduced image or thumbnail (T) obtained from the reference image (10′) supplied by the head (3′) of the endoscope and processed by the current approximated transformation.

37. Process according to claim 36, wherein visual elements or a piece of visual information is/are selected from the group that is formed by visual patterns, image reductions and the parts or zones of images that are obtained respectively from the reference image (10), in the form of an initial image or basic image supplied by the optical system (4) or a real image of the anatomical environment of the target acquired by selection, and the current image (10′) supplied by the optical system (4), if necessary after suitable processing of the latter.

38. Process according to claim 36, wherein it consists in implementing a minimization algorithm that produces a parametric minimization of the quadratic error between the visual elements or piece of visual information (T* and T) obtained respectively from the reference image (10) and from the current image (10′), whereby the approximated transformation is a plane transformation, for example, of homographic type.

39. Process according to claim 36, wherein it consists in implementing a minimization algorithm that produces a statistical minimization of a measure of dissimilarity between histograms that are obtained from visual elements or a piece of visual information (T* and T) obtained respectively from the reference image (10) and from the current image (10′), whereby the approximated transformation consists of a translation in the image plane and a zoom.

40. Process according to claim 36, wherein the movements and the positioning of the end segment (3′) are monitored by two independent actuators (8 and 8′) each associated with one of the two perpendicular directions corresponding to two directions of the plane current image (10′) supplied repeatedly by the head of the endoscope (3′).

41. Process according to claim 36, wherein it comprises two successive phases, namely a phase for manual parameterization of a setpoint (s_{ref} and a phase for automatic video-rate adjustment of the error: e=(s−s_{ref}), where s is the current value that corresponds to the setpoint, toward zero.

42. Process according to claim 41, wherein the parameterization phase consists in, for the practitioner, determining the setpoint (s_{ref}) of the control loop (9) in the form of suitable visual indices that are selected from a reference image (10) for the purpose of monitoring the different degrees of freedom of the flexible endoscope, in particular its free end (3).

43. Process according to claim 41, wherein the adjustment phase consists in repeating the following stages in a cyclic manner:
a) Extraction in the current image (10′) of current visual indices (s) corresponding to visual indices (s_{ref}) forming the setpoint and selected from the reference image (10);
b) Calculation of the error e=(s−s_{ref});
c) Calculation of the control signals to be applied to the actuators (8 and 8′), in particular speed signals, to reduce the error (e);
d) Dispatch of control signals;
e) Acquisition of a new current image (10′) by means of the optical system and optional processing of this image;
f) Return to stage a).

44. Process according to claim 36, wherein it consists in installing in advance an artificial reference in the immediate vicinity of the anatomical target.
